(19) United States
(12) Patent Application Publication
(10) Pub. No.: US 2018/0078406 A1
(43) Pub. Date: Mar. 22, 2018

(54) CONDOM HAVING ONE OR MORE PORTALS

(71) Applicant: Hong Wang, Staten Island, NY (US)

(72) Inventor: Hong Wang, Staten Island, NY (US)

(21) Appl. No.: 15/296,214

(22) Filed: Oct. 18, 2016

Related U.S. Application Data
(63) Continuation-in-part of application No. 15/273,117, filed on Sep. 22, 2016, now abandoned.

(51) Int. Cl. A61F 6/04

(52) U.S. Cl. CPC A61F 6/04; A61F 2006/048

(57) ABSTRACT

A contraceptive device for preventing pregnancy while at the same time providing greater sensation than a traditional condom. The condom includes at least one portal located in the body of the condom.

### RELATED APPLICATIONS

This application is a continuation in part of application Ser. No. 15/273,117 filed on Sep. 22, 2016. Which is herein incorporated by reference in its entirety.

### TECHNICAL FIELD

The present disclosure relates generally to and in particular to condoms with openings therein.

### BACKGROUND

A condom is used as a barrier between the penis and the vagina, in order to prevent sperm that leaves the urethra from entering into the vaginal cavity and thereby transferring disease or impregnating a woman. However, condoms are frequently not used due to loss of sensation.

### SUMMARY

Provided is a contraceptive device for preventing pregnancy, while at the same time providing greater sensation than a traditional condom.

According to one aspect, there is provided a condom, including a solid tip, a base, and a body. The body extends circumferentially and in parallel to a largest circumference of the solid tip. The body extends between and connecting the solid tip and the base. The body includes at least one portal.

Alternatively or additionally, the body includes a plurality of portals.

Alternatively or additionally, the plurality of portals are evenly spaced across the body.

Alternatively or additionally, the plurality of portals are located on a preferential portion of the body and the preferential portion of the body extends between the solid tip and the base.

Alternatively or additionally, the body comprises a cylinder, the preferential portion of the body is located on a curved face of the cylinder, and the preferential portion is located on one half of a circumference of the curved face of the body.

Alternatively or additionally, the body comprises a lattice of intersecting cords and the plurality of portals are formed by open spaces between the intersecting cords.

Alternatively or additionally, the lattice of intersecting cords includes raised structures.

Alternatively or additionally, the raised structures form at least one gross raised structure that extends circumferentially around the body.

Alternatively or additionally, at least one of the gross raised structures is parallel to the base.

Alternatively or additionally, the at least one gross raised structure comprises a ring with breaks in the ring caused by one or more of the portals.

Alternatively or additionally, the at least one portal includes a seal defining a boundary between the body and the at least one portal.

Alternatively or additionally, the seal comprises a thickened portion of the body.

Alternatively or additionally, the body and the solid tip define an interior volume, the interior volume is configured to receive a penis, and the seal is configured to prevent leakage of fluid from the interior volume to an exterior volume when the penis is located in the interior volume.

Alternatively or additionally, the seal includes a reservoir configured to retain a fluid, and the reservoir is configured to release at least a portion of the fluid when pressure is applied to the reservoir.

Alternatively or additionally, the fluid is at least one of a spermicide, a germicide, or a lubricant.

Alternatively or additionally, the at least one portal is located adjacent the solid tip.

Alternatively or additionally, the at least one portal is shaped as a heart, diamond, circle, rectangle, star, or alphanumeric character.

Alternatively or additionally, the body and solid tip define an interior volume, the interior volume is configured to receive a penis and the solid tip is configured to receive a head of the penis.

A number of features are described herein with respect to embodiments of the invention; it will be appreciated that features described with respect to a given embodiment also may be employed in connection with other embodiments.

The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 shows a top perspective view of a portal condom, in an embodiment of the disclosed technology.

FIG. 2 shows a bottom perspective view of the portal condom of FIG. 1.

FIG. 3 shows a side elevation view of the portal condom of FIG. 1.

FIG. 4 shows a top plan view of the portal condom of FIG. 1.

FIG. 5 shows a bottom plan view of the portal condom of FIG. 1.

FIG. 6 shows a perspective view of the portal condom of FIG. 1, with a urethra inserted therein.

FIG. 7 shows an alternative embodiment of the portal condom with cross-connected rings, in an embodiment of the disclosed technology.

FIG. 8 shows a second alternative embodiment of the portal condom with diamond connected rings, in an embodiment of the disclosed technology.

FIG. 9 shows a third alternative embodiment of the portal condom with two cords.

FIG. 10 shows a third alternative embodiment of the portal condom with one cords.

FIG. 11 shows an embodiment of the condom including a plurality of portals.

FIG. 12 shows an embodiment of the condom including a single portal.

FIG. 13 shows an embodiment of the condom including a plurality of portals located in a preferential portion.

### DETAILED DESCRIPTION

Provided is a contraceptive device for preventing pregnancy while at the same time providing greater sensation than a traditional condom.

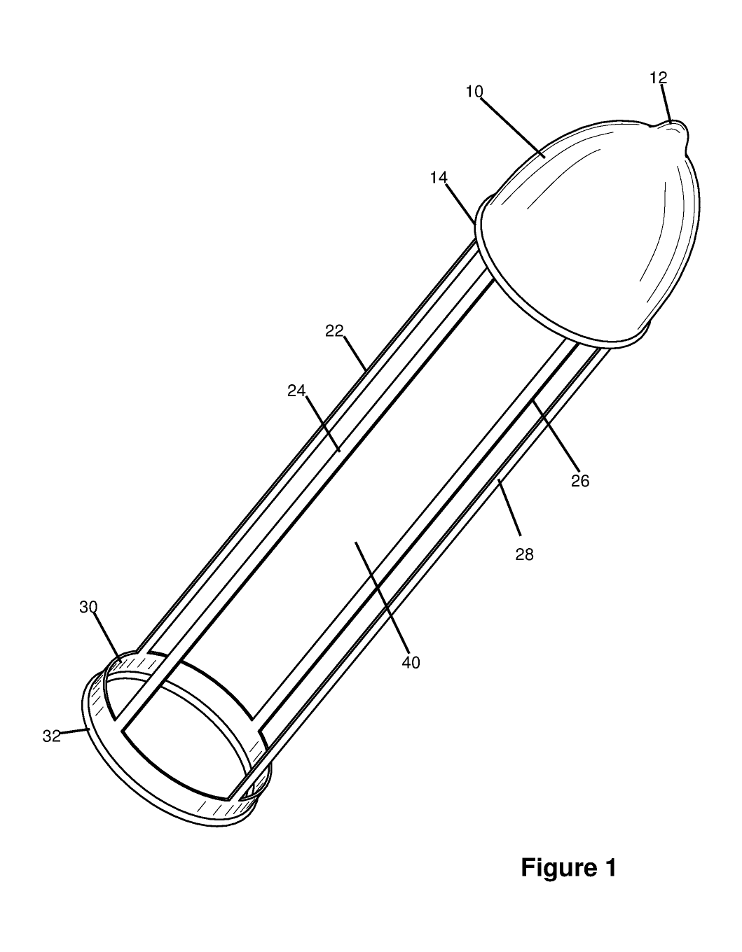

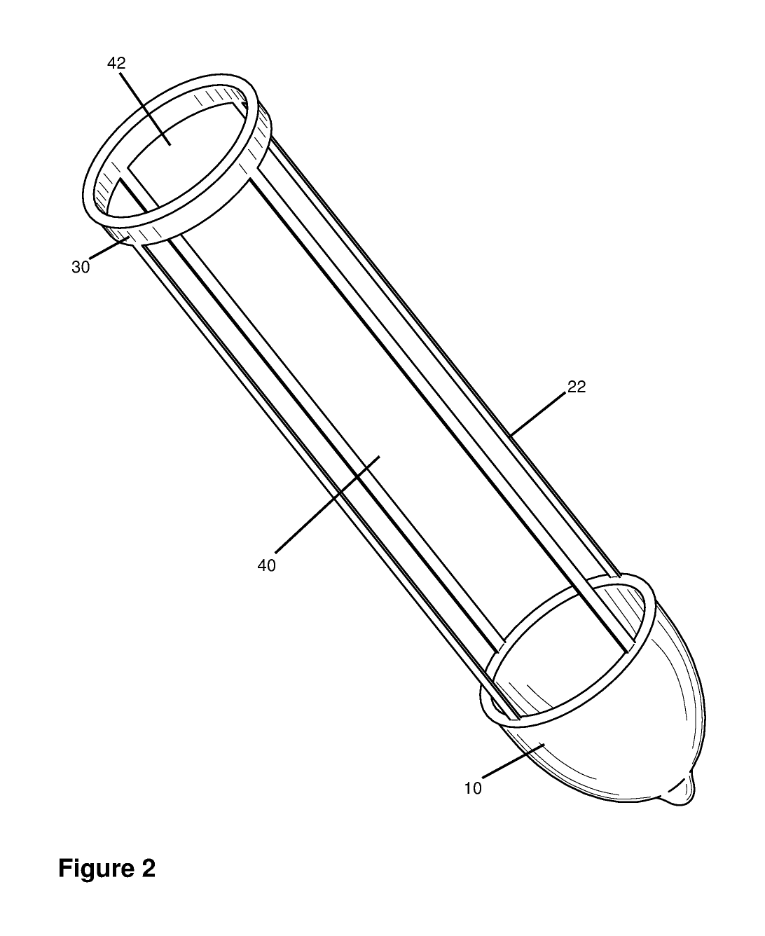

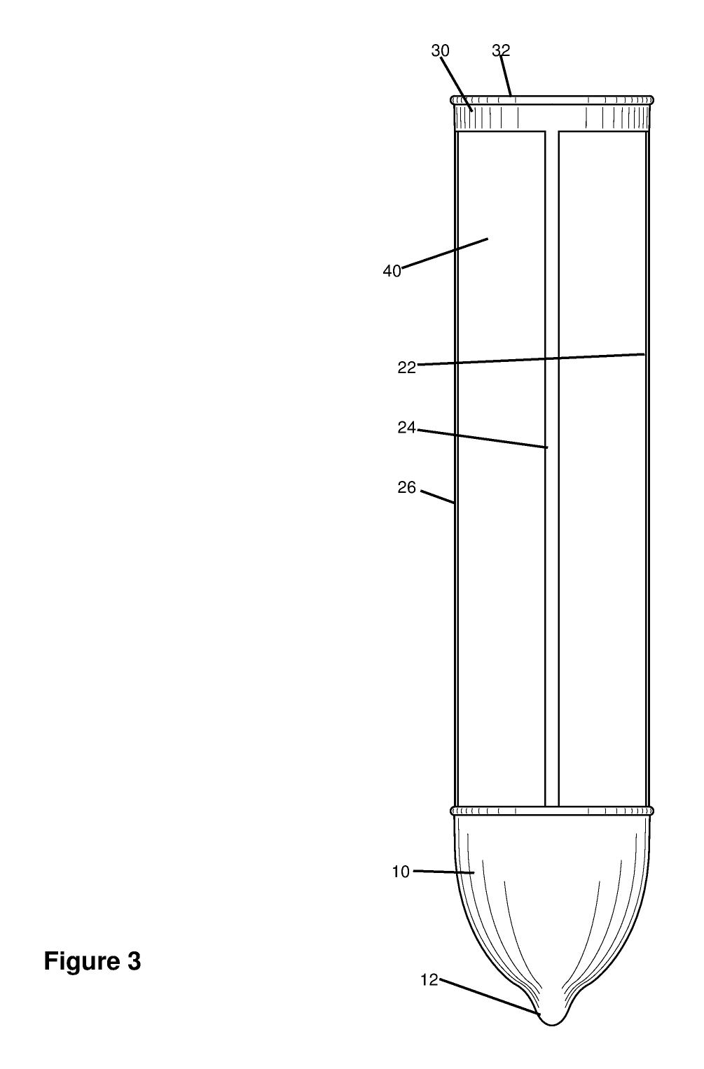

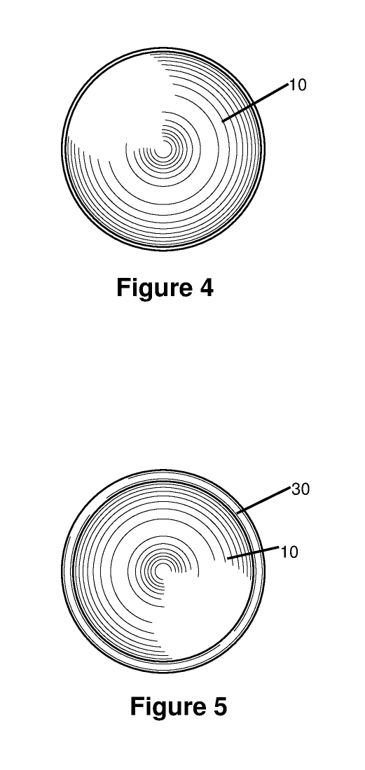

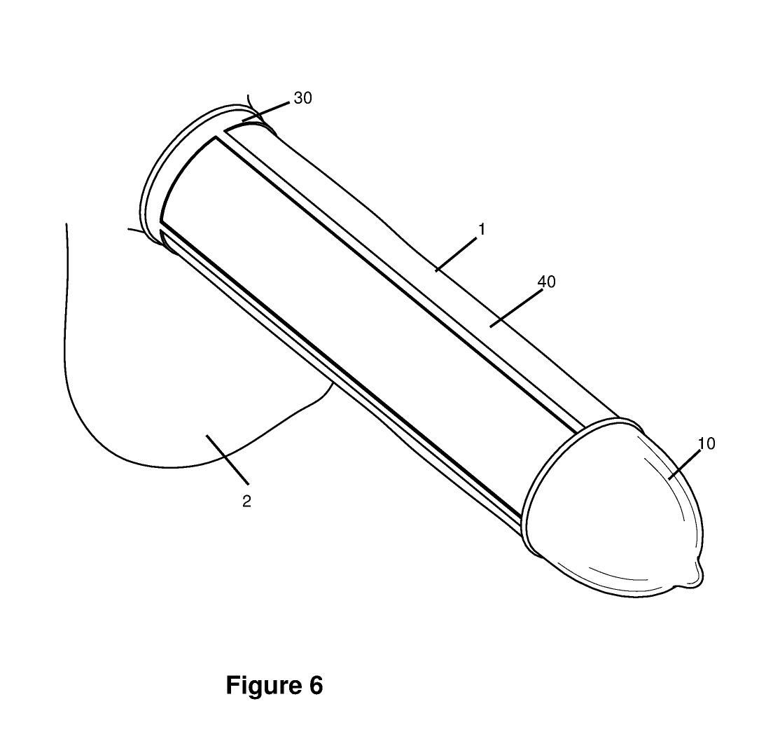

FIGS. 1-5 will be discussed in conjunction with each other. FIG. 1 shows a top perspective view of a portal condom, in an embodiment of the disclosed technology. FIG. 2 shows a bottom perspective view of the portal condom of FIG. 1. FIG. 3 shows a side elevation view of the portal condom of FIG. 1. FIG. 4 shows a top plan view of the portal condom of FIG. 1. FIG. 5 shows a bottom plan view of the portal condom of FIG. 1. The tip 10 of the condom has a reservoir 12 and lowest circumferential ring/largest circumference 14 of the tip. The exterior circumference of the lowest circumferential ring is larger than the interior circumference, and can also be larger on the exterior side, at a portion which is the same interior circumference as other lateral cross-sections thereof. The tip 10 is adapted, configured, designed to, or does cover a tip of a urethra. The tip of a urethra can simply be a portion thereof which it covers extending from an extreme tip where a portal into the penis exists, or can be a widest “mushroom cap” section of the penis, known as the glans penis and/or corona.

Cords may be elongated elements that connect a tip 10 to a base ring 30. In the embodiment shown in FIG. 1, there are four equisized and equispaced cords from one another, cords 22, 24, 26, and 28. A circular base ring 30 with a lip 32 (the lip having a slightly wider exterior circumference) is also shown. The base ring forms a circular portal where the penis tip enters before reaching the tip 10 of the condom. Thus, an interior space 40 is enclosed by the tip 10, cords 22, 24, 26, and 28, and base ring 30. When the penis is inserted into the condom, the portal in the base ring 30 is sealed while the portals (also numbered 40 as the arrow points to the same place—the open space) between the cords allow the penis to be open to the outside and subject to abrasive forces against another object, such as a vaginal wall.

FIG. 4 shows a top plan view of the portal condom of FIG. 1. FIG. 5 shows a bottom plan view of the portal condom of FIG. 1. From the top and bottom view, one can see the interior side of the tip 10. The tip 10 and base 30 remain constant throughout many of the variations of the disclosed technology, where the cord number is changed or diagonals are added.

FIG. 6 shows a perspective view of the portal condom of FIG. 1 with a urethra inserted therein. Here, the urethra 1 fills the interior space of the condom but is exposed through each portal 40 of the condom, the portals being those which are broken by a cord or cords. In some embodiments, the base ring 30 abuts the rest of a person's body 2.

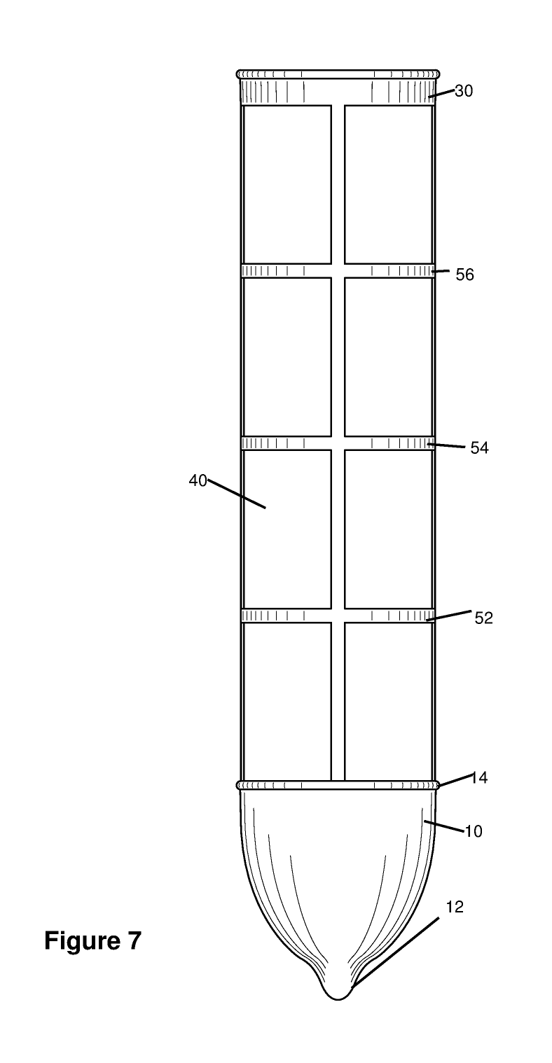

FIG. 7 shows an alternative embodiment of the portal condom with cross-connected rings, in an embodiment of the disclosed technology. The device is as shown with respect to prior figures, except that lateral rings 52, 54 and 56 are shown. Any number of lateral rings can be added. These rings are perpendicular to the cords. For example, lateral ring 54 is perpendicular to cord 22. These lateral rings add stability to the condom, while still allowing the majority of the urethra to be exposed to the exterior environment. Each lateral ring 54 splits a larger portal 40 into smaller portals.

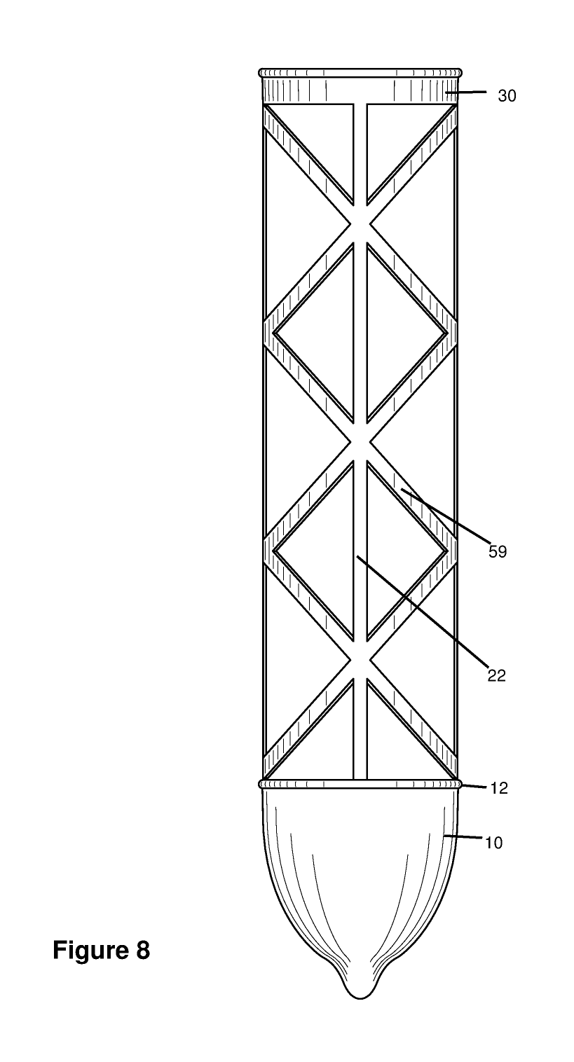

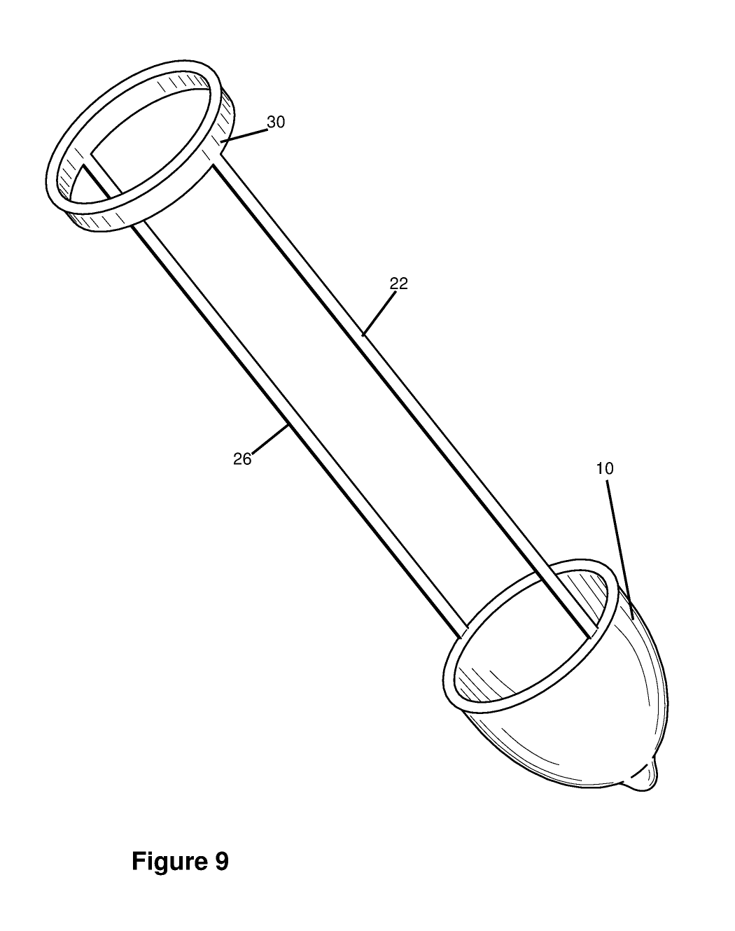

FIG. 8 shows a second alternative embodiment of the portal condom with diamond connected rings, in an embodiment of the disclosed technology. In this embodiment, triangular portals are created as shown. A hypotenuse of each triangle, in some embodiments, is created by a cord such as cord 22, and the legs are created by diagonals 59 which run at 45 degree angles, or another acute angle to one or both of the tip 10 and a cord. This can create a diamond shaped pattern, as shown in the figure.

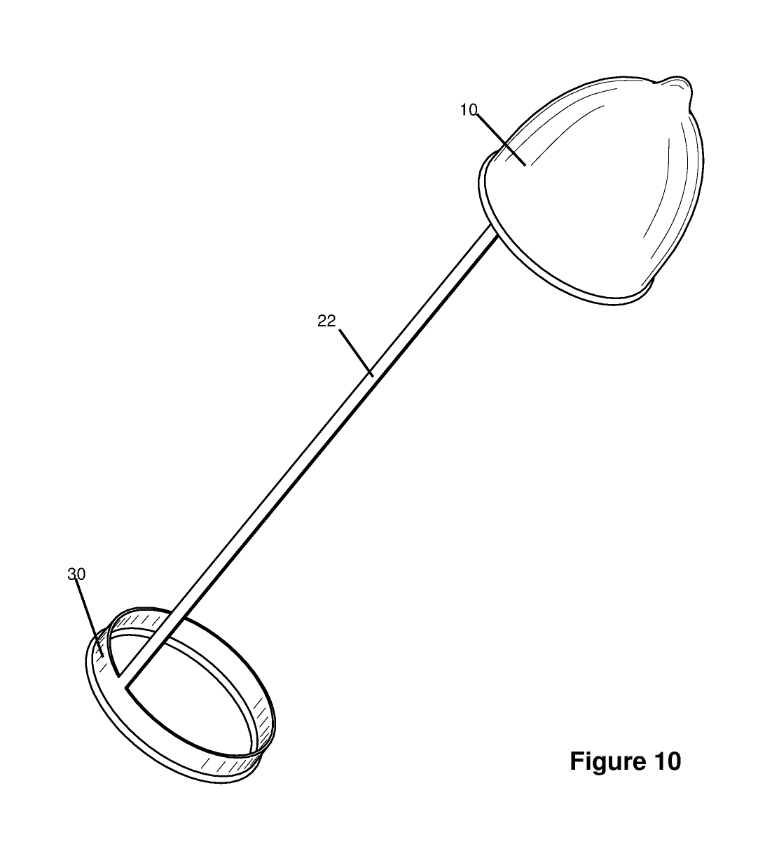

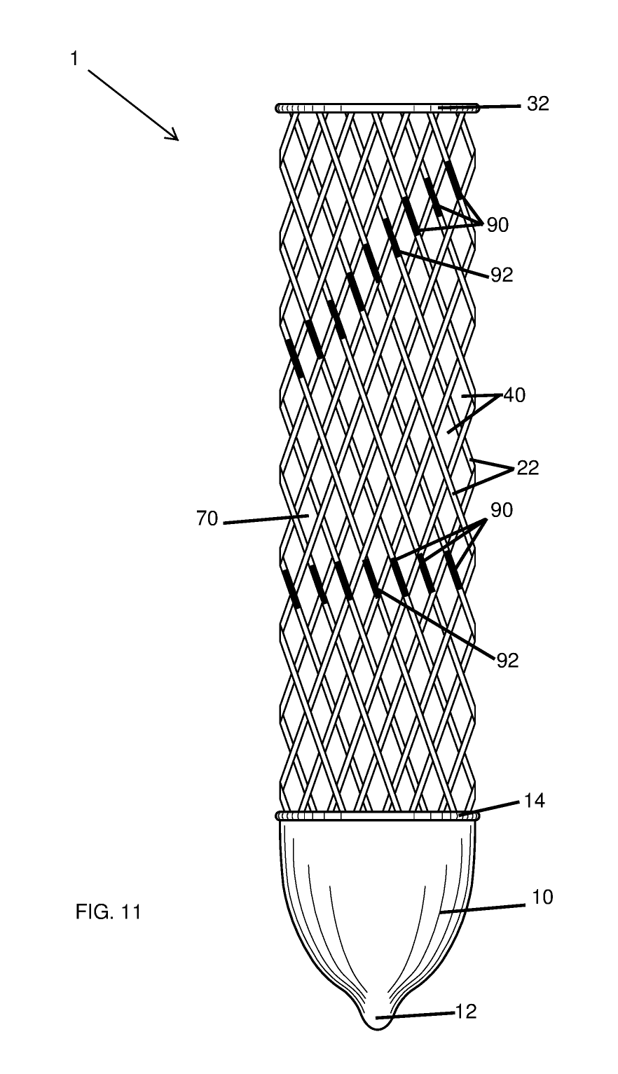

FIG. 9 shows a third alternative embodiment of the portal condom with two cords. FIG. 10 shows a third alternative embodiment of the portal condom with one cords. In these embodiments, the tip 10 and one cord 22, or two cords 22 and 26, are shown connecting the tip 10 to the base 30.

Turning to FIG. 11, a condom 1 including a tip 10, a base 32, and a body 70 is shown. The tip 10 may be a solid tip. The base 32 extends circumferentially and is parallel to the largest circumference 14 of the solid tip 10. The body 70 extends between and connects the tip 10 and the base 32. The body 70 includes at least one portal 40.

“Solid” refers to the tip being impermeable to liquids, in particular water and biological fluids such as semen. As will be understood by one of ordinary skill in the art, the tip 10, body 70, and/or base 32 may be made from latex, lambskin, polyurethane, polyisoprene, or any suitable material.

As shown in FIGS. 11 and 13, the body 70 may include a plurality of portals 40. The portal(s) 40 are openings in the body 70. For example, the portals 40 provide an opening from an interior volume of the body 70 to an exterior of the body 70. The portals 40 may be evenly spaced across the body 70 as shown in FIG. 11. Alternatively, the portals 40 may be located on a preferential portion 90 (represented by a dotted line) of the body 70 as shown in FIG. 13. The preferential portion 90 of the body may extend between the solid tip 10 and the base 32. For example, as shown in FIG. 13, the body 70 may comprise a cylinder and the preferential portion of the body 70 may be located on a curved face of the cylinder. The preferential portion 90 may be located on one quarter, one third, one half, or any other suitable portion of a circumference of the curved face of the body 70.

The tip 10 may be configured to direct fluid away from the preferential portion 90 of the condom (i.e., away from the portals 40). For example, the tip 10 may include fluid channels or be shaped to direct fluid away from the preferential portion 90.

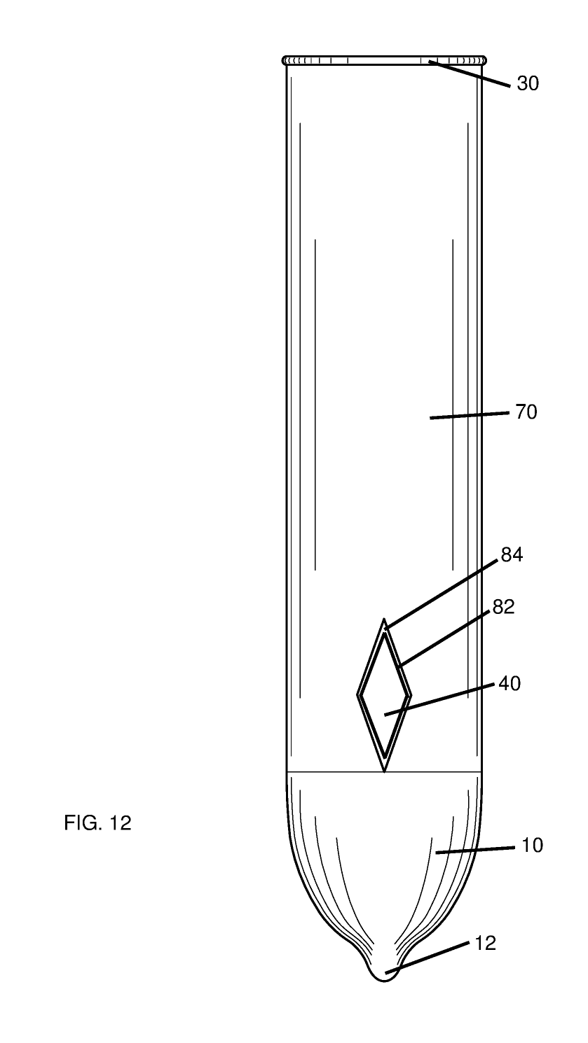

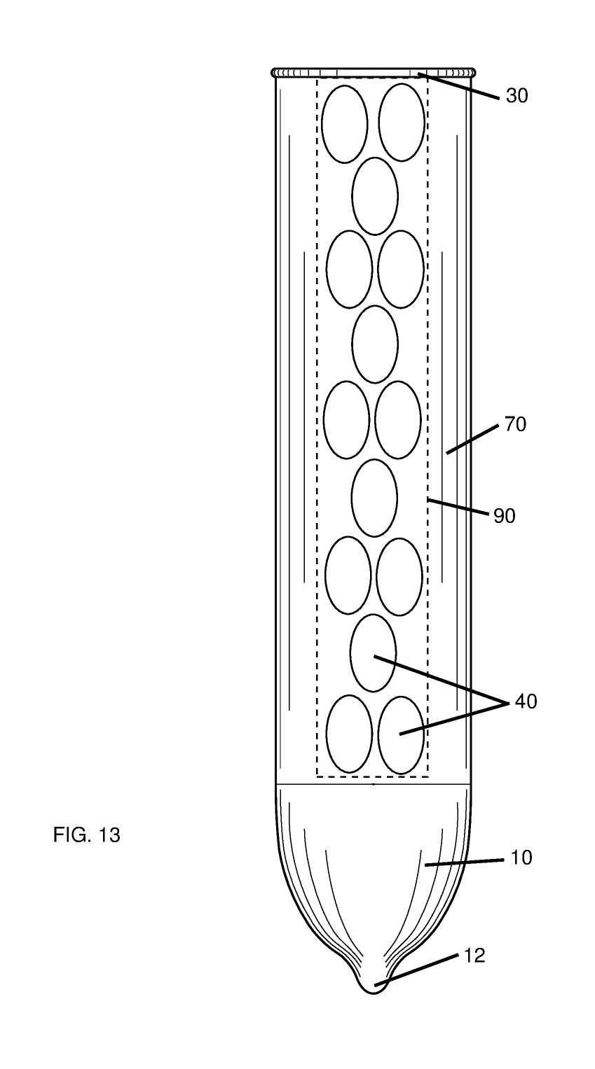

As shown in FIG. 12, the body 70 may include a single portal 40. For example, the portal 40 may be located adjacent the tip 10. The portal(s) 40 may take any suitable shape. For example, the portal(s) 40 may be shaped as a heart, diamond, circle, rectangle, star, or alphanumeric character. As an example, the body 70 may include a series of alphanumeric characters spelling out a word or phrase.

The body 70 may include a lattice of intersecting cords 22 as shown in FIG. 11. The plurality of portals 40 may be formed by the open spaces between the intersecting cords 22. As will be understood by one of ordinary skill in the art, the lattice of intersecting cords 22 may be formed as single individual cords 22 arranged in a pattern with individual cords 22 passing under or over other cords 22. Alternatively, the lattice of intersecting cords 22 may be formed as a single piece shaped in a pattern. For example, the lattice of intersecting cords 22 may be cut from a single piece of latex.

As shown in FIG. 11, the lattice of intersecting cords 22 may also include raised structures 90. The raised structures 90 may form at least one gross raised structure 92. For example, the gross raised structure 92 may extend circumferentially around the body 70. The gross raised structure(s) 92 may be parallel to the base 32. Alternatively or additionally, the gross raised structure(s) 92 may form a ring with breaks in the ring caused by one or more of the portals 40.

FIG. 11 shows two gross raised structures 92. The gross raised structure 92 located towards the base 32 of the body 70 forms a ring around the body 70 that is not parallel to the base 32. The gross raised structure 92 located towards the tip 10 forms a ring around the body 70 and is parallel to the base 32. As will be understood by one of ordinary skill in the art, the gross raised structure 92 being parallel to the base 32 does not require that the gross raised structure 92 be perfectly parallel to the base 32. As an example, the gross raised structure 92 being parallel to the base 32 may include the gross raised structure 92 having an angle of less than 5° or less than 10° from a plane defined by the base 32.

Turning to FIG. 12, the at least one portal 40 may include a seal 82 defining a boundary between the body 70 and the at least one portal 40. The seal 82 may comprise a thickened portion of the body 70. For example, the body 70 and the tip 10 may define an interior volume configured to receive a penis. The seal 82 may be configured to prevent a leakage of fluid (e.g., semen) from the interior volume to an exterior volume when the penis is located in the interior volume of the condom. That is, when the condom is worn by a user, the seal 82 may be configured to prevent leakage from the inside of the condom to an outside of the condom.

The seal 82 may include a reservoir 84. The reservoir 84 may be configured to retain a fluid and to release at least a portion of the fluid when pressure is applied to the reservoir. For example, the reservoir 84 may be configured to release a portion of the fluid maintained in the reservoir 84 during sex. The fluid retained in the reservoir 84 may be at least one of a spermicide, a germicide, or a lubricant.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

The phrase “and/or” means “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” has the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, means at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are open-ended, i.e., meaning including but not limited to.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application

1. A condom, comprising:
a solid tip;
a base extending circumferentially and in parallel to a largest circumference of the solid tip; and
a body extending between and connecting the solid tip and the base, the body including at least one portal.

2. The condom of claim 1, wherein the body includes a plurality of portals.

3. The condom of claim 2, wherein the plurality of portals are evenly spaced across the body.

4. The condom of claim 2, wherein the plurality of portals are located on a preferential portion of the body and the preferential portion of the body extends between the solid tip and the base.

5. The condom of claim 4, wherein the body comprises a cylinder, the preferential portion of the body is located on a curved face of the cylinder, and the preferential portion is located on one half of a circumference of the curved face of the body.

6. The condom of claim 2, wherein the body comprises a lattice of intersecting cords and the plurality of portals are formed by open spaces between the intersecting cords.

7. The condom of claim 6, wherein the lattice of intersecting cords includes raised structures.

8. The condom of claim 7, wherein the raised structures form at least one gross raised structure that extends circumferentially around the body.

9. The condom of claim 8, wherein at least one of the gross raised structures is parallel to the base.

10. The condom of claim 8, wherein the at least one gross raised structure comprises a ring with breaks in the ring caused by one or more of the portals.

11. The condom of claim 1, wherein the at least one portal includes a seal defining a boundary between the body and the at least one portal.

12. The condom of claim 11, wherein the seal comprises a thickened portion of the body.

13. The condom of claim 12, wherein the body and the solid tip define an interior volume, the interior volume is configured to receive a penis, and the seal is configured to prevent leakage of fluid from the interior volume to an exterior volume when the penis is located in the interior volume.

14. The condom of claim 11, wherein the seal includes a reservoir configured to retain a fluid, and the reservoir is configured to release at least a portion of the fluid when pressure is applied to the reservoir.

15. The condom of claim 14, wherein the fluid is at least one of a spermicide, a germicide, or a lubricant.

16. The condom of claim 1, wherein the at least one portal is located adjacent the solid tip.

17. The condom of claim 16, wherein the at least one portal is shaped as a heart, diamond, circle, rectangle, star, or alphanumeric character.

18. The condom of claim 1, wherein the body and solid tip define an interior volume, the interior volume is configured to receive a penis and the solid tip is configured to receive a head of the penis.
